(19) United States
(12) Patent Application Publication
(10) Pub. No.: US 2024/0390634 A1
(43) Pub. Date: Nov. 28, 2024

(54) Methods of Reducing the Risk of Mortality Associated With a Medical Treatment

(71) Applicant: Mallinckrodt Pharmaceuticals Ireland Limited, Dublin (IE)

(72) Inventors: Jim Potenziano, Binghamton, NY (US); Joe Stauffer, Skillman, NJ (US)

(73) Assignee: Mallinckrodt Pharmaceuticals Ireland Limited, Dublin (IE)

(21) Appl. No.: 18/639,099

(22) Filed: Apr. 18, 2024

Related U.S. Application Data
(63) Continuation of application No. 15/854,643, filed on Dec. 26, 2017, now Pat. No. 11,992,620.
Application No. 15/854,643 is a division of application No. 14/313,767, filed on Jun. 24, 2014, now abandoned.
(60) Provisional application No. 61/839,352, filed on Jun. 25, 2013.

(51) Int. Cl. A61M 16/12; A61K 33/00; A61M 16/00; A61M 16/10; G16H 10/20; G16H 50/30

(52) U.S. Cl. CPC A61M 16/12; A61K 33/00; A61M 16/0096; G16H 10/20; G16H 50/30; A61M 2016/1035; A61M 2202/0208; A61M 2202/025; A61M 2202/0275; Y02A 90/10

(57) ABSTRACT

Disclosed are methods of treatment that permit a reduction of risk of mortality in infants who are candidates for treatment with inhaled nitric oxide, by identifying a subset of such infants who are at an increased risk of mortality upon treatment with inhaled nitric oxide; also disclosed are related systems for use in administering inhaled nitric oxide and methods of distributing a pharmaceutical product.

### CLAIM OF PRIORITY

This application is a continuation of and claims priority to U.S. application Ser. No. 15/854,643, filed on Dec. 26, 2017 (U.S. Pat. No. 11,992,620), which is a divisional and claims priority to U.S. application Ser. No. 14/313,767, filed on Jun. 24, 2014 (abandoned), which claims priority under 35 USC § 119(e) to U.S. Application Ser. No. 61/839,352, filed on Jun. 25, 2013. The entire contents of the prior applications are hereby incorporated by reference.

### TECHNICAL FIELD

The present invention relates to methods for reducing the incidence of mortality in infants undergoing medical treatment.

### BACKGROUND

Bronchopulmonary dysplasia (BPD) is a chronic lung disease (CLD) of pre-term neonates, attributable to lung immaturity with associated surfactant deficiency, systemic inflammation, and the adverse effects of ventilator and supplemental oxygen therapy for acute respiratory problems. BPD is defined clinically as the need for supplemental oxygen at 36 weeks' postmenstrual age (PMA) (±3 days). There are an estimated 10,000 to 15,000 pre-term neonates diagnosed with BPD in the United States (US) each year. The risk of developing BPD is related to the degree of prematurity, with the highest risk of BPD occurring in pre-term neonates born prior to 26 weeks gestation and ≤750 g body weight at birth. Pre-term neonates who develop BPD may consequently need long-term breathing support, such as with the use of nasal continuous positive airway pressure (NCPAP) machines or ventilators. Prediction of which pre-term neonates are considered to be at risk of developing BPD can be done by standard methods, e.g., as described in Laughon et al., (2011) Am J Respir Crit Care Med 183:1715-1722.

BPD can develop for several reasons. Pre-term neonates—especially those who develop respiratory distress syndrome (RDS)—frequently require supplemental oxygen therapy (oxygen given through nasal prongs, a mask, or a breathing tube). Preterm neonates with RDS require mechanical ventilation, but over-ventilation of this patient population often results in BPD, due to injury to the lungs. See Northway et al., (1967) New Engl J Med 276:357-368. Infections that occur before or shortly after birth also can contribute to BPD. Lack of lung maturation can also affect a pre-term neonate's chance of developing BPD. Pre-term neonates as young as 22 weeks gestational age are surviving, but generally exhibit immature lungs, with large and simplified alveoli with dysmorphic capillaries, negligible airway epithelial lesions, and mild interstitial fibroproliferation. The younger the pre-term neonate, the less-developed are his or her lungs, leaving him or her more likely to develop BPD.

Pre-term neonates with BPD are at greater risk for poor developmental outcomes, acute and chronic lung disease, an increased susceptibility to infections, and more frequent hospitalization for respiratory illness during the first year of life. Antenatal infections, such as chorioamnionitis and acquired pneumonias, are additional potentially complicating and contributing factors in the development of BPD. Late clinical sequelae of BPD result in recurrent respiratory hospitalizations (50% in the first year) and frequent emergency room visits, pulmonary hypertension, late asthma and structural airway disease, exercise intolerance, apnea, sudden death, systemic hypertension, left ventricular hypertrophy, growth failure, feeding difficulties, and developmental delays.

Currently, there are no approved medicinal products in the US or globally to prevent the development of BPD in pre-term neonates. Following FDA approval of inhaled nitric oxide (a potent and selective pulmonary vasodilator) for the treatment of pulmonary hypertension secondary to hypoxic respiratory failure (HRF) in the term/near term neonate, it has been suggested that inhaled nitric oxide might also benefit the sick pre-term neonate and, in particular, reduce the incidence and/or severity of BPD. Radiographic evidence suggests prolonged inhaled nitric oxide therapy improves lung vascular growth after VEGF inhibition and improves alveolarization in experimental BPD in preterm lambs. The potential therapeutic targets for the use of inhaled nitric oxide in the pre-term neonate include: improved gas exchange and reduced FiO₂, lower pulmonary artery pressure, anti-inflammatory and antioxidant effects, sustained surfactant production, and preservation or stimulation of angiogenesis and alveolarization in the developing lung.

Several studies have been directed to use of inhaled nitric oxide to prevent (reduce the incidence of) BPD.

Kinsella et al., (1999) Lancet 354:1061-1065, was a randomized, double-blind, placebo-controlled, multi-center study in 80 pre-term neonates that was terminated early for enrollment issues. Inhaled nitric oxide was administered at a dose of 5 ppm for 7 days. The results of this study demonstrated a 15.4% reduction in death or BPD (77% inhaled nitric oxide vs 91% placebo; p=0.14), with no increase in severe intraventricular hemorrhage (IVH).

Schreiber et al., N Engl J Med (2003) 349:2099-2107, was a randomized, double-blind, placebo-controlled, single-center study in 207 pre-term neonates administered a starting dose of inhaled nitric oxide 10 ppm for 1 day, then 5 ppm for 6 days. The results of the Schreiber study demonstrated a 24% reduction in CLD/death (48.6% inhaled nitric oxide vs 63.7% placebo; p=0.03), a 47% reduction in severe IVH/PVL (PVL=periventricular leukomalacia) (12.4% inhaled nitric oxide vs 23.5% placebo; p=0.04), and a 47% reduction in “abnormal or delayed” development (p=0.01).

Van Meurs et al., Pediatrics (2004) 113:559-564, was a randomized, double-blind, placebo-controlled, multi-center study in 420 (800 planned) pre-term neonates. Inhaled nitric oxide was administered at a starting dose of 5 ppm and increased to 10 ppm if no response (Mean 3 days therapy). The study was stopped prematurely. The results of this study showed overall no difference vs placebo in death or BPD and no difference in IVH.

Kinsella et al., N Engl J Med (2006) 355:354-364, designated the “INOT 25” study, was a randomized, double-blind, placebo-controlled, multi-center study in 793 subjects. Inhaled nitric oxide was administered at a dose of 5 ppm while the patient was intubated, with a median treatment duration of 14 days. The results of the INOT 25 study showed overall no difference vs placebo in CLD or death (71.6% vs 75.3%) and a 27% reduction in IVH/PVL (17.5% vs 23.9%).

Mercier et al., Lancet (2010) 376:346-54, designated the “INOT 27” study, was a randomized, double-blind, placebo-controlled, multi-center study in 35 centers in nine European Union countries (800 subjects). Inhaled nitric oxide was administered at a dose of 5 ppm for 7-21 days (treatment duration Mean 16.3 days). The results of the INOT 27 study showed overall no difference in survival without BPD at 36 weeks postmenstrual age (placebo 65.5% vs inhaled nitric oxide 65% (p=0.73)).

Ballard et al., N Engl J Med (2007) 357:1444-1445, and Ballard et al., N Engl J Med (2006) 355:343-353, report a randomized, double-blind, placebo-controlled, multicenter study in which 537 pre-term neonates received either inhaled nitric oxide or placebo. Therapy was initiated between Day 7 and Day 21. Patients were administered inhaled nitric oxide 20 ppm for 2 to 4 days, then stepped-down in weekly intervals. All patients were treated for 24 days. The results of this study showed a 20% improvement in survival without CLD at 36 weeks postmenstrual age with inhaled nitric oxide (inhaled nitric oxide 43.9% vs placebo 36.8%, p=0.042). Patients were discharged (p=0.04) or off all ventilator support sooner on study drug (40 weeks, inhaled nitric oxide 65% vs placebo 53%, p=0.01; 44 weeks, inhaled nitric oxide 82% vs placebo 76%, p=0.03).

### SUMMARY

It has now been discovered that a particular subset of patients is at an increased risk of mortality when treated with inhaled nitric oxide, and that this subset is defined in part by one or more of race, gender, and gestational age, and also by determination of risk of developing BPD. The affected subset of patients is white, male, pre-term neonates; more specifically, white, male, pre-term neonates less than 27 weeks gestational age; and even more specifically, white, male, pre-term neonates less than 27 weeks gestational age that are at risk of developing BPD. The same risk was not observed in pre-term neonates (male or female) of other races, specifically American Indian/Alaska Native, Asian, black, and Hispanic pre-term neonates, nor in female pre-term neonates of any race (including white), nor in neonates of gestational age greater than 27 weeks. Non-white pre-term neonates as a class and female pre-term neonates as a class, even those of gestational age less than 27 weeks, were able to safely receive inhaled nitric oxide administered as a means to prevent development of BPD. The newly discovered risk can be described as follows: in white males <27 weeks gestational age, inhaled nitric oxide may have an increased risk of death as compared to other subgroups. Another way to describe the risk is: in white male pre-term infants <27 weeks gestational age, without pulmonary hypertension and at risk of developing BPD, inhaled nitric oxide may have an increased risk of death as compared to other subgroups. There was seen in one study an imbalance in deaths in this subgroup when treated with nitric oxide as compared to placebo at doses of 20 ppm for 3 days reduced to 10 ppm for 10 days and 5 ppm for remainder of 24 days. This was not seen in other subgroups.

In one aspect, the invention is directed to a method of treatment that includes identifying a plurality of neonates (i.e., two or more) as being candidates for treatment with inhaled nitric oxide (preferably for a reason—such as prevention of BPD—other than treatment of a condition involving pulmonary hypertension); determining that the race of a first neonate of the plurality is black or another non-white race such as American Indian/Alaska Native, Asian, or Hispanic; administering inhaled nitric oxide to the first neonate; determining that the race of a second neonate of the plurality is white; determining that the gender of the second neonate is male; determining that the second neonate is of less than 27 weeks gestational age; determining that the fact that the second neonate is a white male of less than 27 weeks gestational age means the second neonate is at a higher risk of mortality upon administration of inhaled nitric oxide compared to a black (or other non-white) male neonate or a female neonate of the same gestational age; and excluding the second neonate from treatment with inhaled nitric oxide, based on the determination that the second neonate has the higher risk of mortality. Thus, neonates who are determined not to have that higher risk of mortality (e.g., because they are non-white, or are female, or are not less than 27 weeks gestational age) are treated with inhaled nitric oxide, while those who have the higher risk of mortality are not treated with inhaled nitric oxide. The reason that the neonates are candidates for treatment with inhaled nitric oxide may be that they are at risk for developing BPD. The reason is preferably not that the neonates have a condition associated with pulmonary hypertension, since such a condition may itself be acutely life-threatening and, in the judgment of the physician, require treatment with inhaled nitric oxide even where the neonate is a white male of less than 27 weeks gestational age. Thus, a further determination that may be made prior to deciding not to treat with inhaled nitric oxide is that the neonate does not have pulmonary hypertension.

In another aspect, the second neonate is identified (e.g., diagnosed) as being at risk of incurring BPD, e.g., from damage attributable to supplemental oxygen therapy that is frequently given to pre-term neonates. Such a neonate may be excluded from treatment of inhaled nitric oxide at a dosing regimen typically used to prevent BPD because of the mortality risk specific to white, male, pre-term neonates of less than 27 weeks gestational age.

Administration of inhaled nitric oxide to pre-term neonates who are candidates for inhaled nitric oxide therapy because they have been identified as being at risk for developing BPD typically begins no less than five days after birth, due at least in part to risk of brain hemorrhage. Treatment of these pre-term neonates may begin at a point during the period that is 5 to 14 days after birth, 5 to 10 days after birth, 5 to 21 days after birth, 7 to 21 days after birth, or 5 to 7 days after birth. Administration of inhaled nitric oxide for the purpose of preventing BPD in susceptible pre-term neonates generally continues for 20-30 days after it is initiated. In specific treatment protocols, administration of inhaled nitric oxide may be continued for at least 18 days, 19 days, 20 days, 21 days, 22 days, 23 days, 24 days, 25 days, 26 days, 27 days, 28 days, 29 days or 30 days, and may involve a stepwise decrease in nitric oxide concentration (e.g., in two, three, or four steps) over that time period. Treatments requiring inhaled nitric oxide for prevention or treatment of a different disorder or underlying condition may have a different dosing regimen.

The second neonate may have an underlying condition, such as pulmonary hypertension or hypoxic respiratory failure, that may benefit from inhaled nitric oxide treatment at a very different dosing regimen than suggested for prevention of BPD. Thus, in one aspect, a neonate who is male, white, less than 27 weeks gestational age, and at risk of BPD may indeed safely receive inhaled nitric oxide for the underlying condition, but for a limited duration less than that typically utilized to prevent BPD (e.g., a limited duration of approximately one, two, three, four, five, or six hours, 12 hours, 18 hours, 24 hours, 36 hours, 48 hours, three days, or four days). The duration will typically be long enough to relieve, at least temporarily, the underlying condition, but will usually be less than 96 hours (e.g., less than 80 hours, or less than 70 hours, or less than 60 hours. In some embodiments, the treatment will last for a period between approximately 40 hours and 70 hours.

One or more of the neonates may have, or be at risk of incurring, pulmonary hypertension that can be relieved at least in part with inhaled nitric oxide at a specific dosing regimen (timing, duration, amount, and manner of dosing) different from that typically used to prevent BPD. The second neonate may be given an alternate acceptable treatment for his/her underlying condition. The second neonate may also be at risk of mortality due to an underlying condition, such pulmonary hypertension, and may be administered inhaled nitric oxide at a specific dosing regimen that is different (much shorter duration) from the dosing regimen that is used for the prevention of BPD. If the underlying condition is hypoxic respiratory failure, the alternate treatment could be, for example, ventilatory support (e.g., high-frequency oscillatory ventilation) with supplemental oxygen, extracorporeal membrane oxygenation (ECMO), intravenous fluids, surfactant, and/or bicarbonate therapy. In some embodiments, the first and/or second neonate is a potential candidate for inhaled nitric oxide at least in part because he/she is identified as being at risk of BPD. In other embodiments, the first and/or second neonate is a candidate for inhaled nitric oxide because the neonate has a condition that can lead to pulmonary hypertension, or is undergoing, or has just undergone, a procedure that can lead to pulmonary hypertension, such as cardiac surgery involving heart/lung bypass.

In another aspect, the method of treatment includes identifying a plurality of neonates as being candidates for treatment with inhaled nitric oxide; determining that the race of a first neonate of the plurality is black or another non-white race or that the gender of the first neonate is female; administering inhaled nitric oxide to the first neonate; determining that the race of a second neonate of the plurality is white; determining that the gender of the second neonate is male; determining that the fact that the second neonate is a white male means the second neonate is at a higher risk of mortality upon administration of inhaled nitric oxide compared to a nonwhite (e.g., black) or female neonate of the same gestational age; and excluding the second neonate from treatment with inhaled nitric oxide, based on the determination that the second neonate has the higher risk. In some embodiments, the neonate is less than 27 week gestational age and/or is at risk of developing BPD. One or more of the neonates may have, or be at risk of incurring, pulmonary hypertension that can be relieved at least in part with inhaled nitric oxide. The second neonate may be given an alternate acceptable treatment (or inhaled nitric oxide at a specific dosing regimen dissimilar from the dosing regimen that would typically be used to prevent BPD) for his/her underlying condition. If the underlying condition is pulmonary hypertension or hypoxic respiratory failure, the second neonate may be given one or more alternate acceptable treatment(s), such as ventilatory support (e.g., high-frequency oscillatory ventilation) with supplemental oxygen, ECMO, intravenous fluids, surfactant, and/or bicarbonate therapy, instead of inhaled nitric oxide. In other embodiments, the second neonate is a candidate for inhaled nitric oxide at a specific dosing regimen different than the dosing regimen administered to the first neonate for prevention of BPD because he has a condition that can lead to pulmonary hypertension, or is undergoing, or has just undergone, a procedure that can lead to pulmonary hypertension, such as cardiac surgery involving heart/lung bypass. In this instance, the dosing regimen of inhaled nitric oxide received by the second neonate is for a shorter duration than would typically be used to prevent BPD.

In another aspect, the method of treatment includes performing at least one diagnostic process (which may include performing echocardiography) to identify a plurality of neonates who have an underlying condition, such as hypoxic respiratory failure or being at risk of BPD, that makes them candidates for inhaled nitric oxide treatment; determining that the race of a first neonate of the plurality is black (or another non-white race) or the gender is female; administering inhaled nitric oxide to the first neonate; determining that a second neonate of the plurality is a white male of less than 27 weeks gestational age; determining that the fact that the second neonate is a white male of less than 27 weeks gestational age means the second neonate is at a higher risk of mortality upon administration of inhaled nitric oxide compared to a black or female neonate patient of the same gestational age; and either providing an alternative treatment to the second neonate or administering inhaled nitric oxide at a shorter duration than what would typically be used for prevention of BPD. Such duration for the second neonate may be less than 1 day, 1 day, 2 days, 3 days, or 4 days, or an increment of hours that is less than 96 hours, e.g., 6 hours, 12 hours, 18 hours, 24 hours, 36 hours, 40 hours, 48 hours, 50 hours, 60 hours, 72 hours, or 84 hours. The second neonate may be given an alternate acceptable treatment for his underlying condition, such as one or more of ventilatory support (e.g., high-frequency oscillatory ventilation) with supplemental oxygen, ECMO, intravenous fluids, surfactant, and/or bicarbonate therapy, instead of inhaled nitric oxide.

Also within the invention is a method of selecting a treatment for a neonate, the method encompassing performing at least one diagnostic process (which may include echocardiography) to identify a neonate (e.g., a premature infant of less than 27 weeks gestational age who may be at risk of BPD) with pulmonary hypertension and/or hypoxic respiratory failure; determining that the neonate is a white male; determining that the fact that the neonate is a white male means he is at a higher risk of mortality upon administration of inhaled nitric oxide compared to a black or other non- white neonate or a female neonate of the same gestational age when administered inhaled nitric oxide in a fashion consistent with preventing BPD; and either excluding the white male neonate from treatment with inhaled nitric oxide or treating the white male neonate with inhaled nitric oxide at a dosing regimen of a significantly shorter duration than the 20-30 days that would typically be used for preventing BPD in pre-term neonates susceptible to BPD, based on the determination that the white male neonate is at a higher risk of mortality than a black neonate or female neonate of the same gestational age if treated with a dosing regimen suitable for preventing BPD. Alternatives to treatment with inhaled nitric oxide for treating or preventing hypoxic respiratory failure or pulmonary hypertension may include one or more of ventilatory support (e.g., with supplemental oxygen), ECMO, intravenous fluids, surfactant, and/or bicarbonate therapy or other acceptable treatment. The ventilatory support may involve high-frequency oscillatory ventilation. In some embodiments, the white male neonate is considered to be at risk of BPD. In other embodiments, the neonate is a candidate for inhaled nitric oxide because he has a condition that can lead to pulmonary hypertension, or is undergoing, or has just undergone, a procedure that can lead to pulmonary hypertension, such as cardiac surgery involving heart/lung bypass.

Also within the invention is a method of selecting a treatment for a pre-term neonate, the method encompassing identifying a pre-term neonate of less than 27 weeks gestational age; treating the pre-term neonate with mechanical ventilation or positive airway pressure support; determining that the pre-term neonate is at risk of BPD; determining that the pre-term neonate is a white male; determining that the fact that the pre-term neonate is a white male means the pre-term neonate is at a higher risk of mortality upon administration of inhaled nitric oxide for prevention of BPD compared to a black or other non-white pre-term neonate or a female pre-term neonate of the same gestational age; and, based on the determination of higher risk, excluding the white, male, pre-term neonate from treatment with inhaled nitric oxide for prevention of BPD. The white, male, pre-term neonate may be treated with high-frequency oscillatory ventilation instead of inhaled nitric oxide.

In another aspect, the method of treatment includes identifying a pre-term neonate of less than 27 weeks gestational age as being in need of treatment for prevention of BPD; determining that the race of the pre-term neonate is white and the gender is male; determining that the fact that the pre-term neonate is a white male means the pre-term neonate is at a higher risk of mortality upon administration of inhaled nitric oxide compared to a black or other non-white pre-term neonate or a female pre-term neonate of the same gestational age; deciding to postpone inhaled nitric oxide treatment until the white, male, pre-term neonate reaches 27 weeks postmenstrual age; administering a treatment other than inhaled nitric oxide to the white, male, pre-term neonate; and, when the white, male, pre-term neonate reaches 27 weeks postmenstrual age, beginning administration of inhaled nitric oxide to the white, male, pre-term neonate, where the decision to postpone inhaled nitric oxide treatment until 27 weeks postmenstrual age is based on the higher risk of mortality in white, male, pre-term neonates of less than 27 weeks postmenstrual age. The nitric oxide will typically be administered initially at 20 ppm, and may be decreased gradually to 10 ppm, then 5 ppm, and finally 2 ppm over the course of treatment. The treatment administered prior to beginning inhaled nitric oxide administration may be, for example, ventilatory support (e.g., with supplemental oxygen).

In another aspect, the method of treatment includes identifying a pre-term neonate of less than 27 weeks gestational age (e.g., less than 26 weeks) as being in need of inhaled nitric oxide for prevention or treatment of a condition involving pulmonary hypertension (e.g., hypoxic respiratory failure); determining that the race of the pre-term neonate is white; determining that the gender of the pre-term neonate is male; determining that the fact that the pre-term neonate is a white male means the pre-term neonate is at a higher risk of mortality upon administration of inhaled nitric oxide compared to a black or other non-white pre-term neonate or a female pre-term neonate of the same gestational age; administering a treatment other than inhaled nitric oxide to the white, male, pre-term neonate; and, when the white, male, pre-term neonate reaches 27 weeks postmenstrual age, beginning administration of inhaled nitric oxide to the white, male, pre-term neonate, where the decision to postpone inhaled nitric oxide treatment until 27 weeks postmenstrual age is because of the higher risk of mortality in white, male, pre-term neonates of less than 27 weeks postmenstrual age. The nitric oxide will typically be administered at 20 ppm. The treatment administered prior to beginning inhaled nitric oxide administration may be, for example, ventilatory support (e.g., with supplemental oxygen), ECMO, intravenous fluids, surfactant, and/or bicarbonate therapy, or another alternative therapy. Such treatment (except ECMO) may optionally be continued during the period that inhaled nitric oxide is administered. The ventilatory support may involve high-frequency oscillatory ventilation. Treatment of any pre-term neonate with ECMO may be possible only if the pre-term neonate's blood vessels are of adequate size. In some embodiments, the white, male, pre-term neonate is considered to be at risk of BPD, in addition to having a condition involving pulmonary hypertension. The pre-term neonate may be a candidate for inhaled nitric oxide because he has a condition that can lead to pulmonary hypertension, or is undergoing, or has just undergone, a procedure that can lead to pulmonary hypertension, such as cardiac surgery involving heart/lung bypass.

In another aspect, the method of treatment includes identifying a pre-term neonate of less than 27 weeks gestational age as being a candidate for treatment with inhaled nitric oxide because of an underlying condition; determining that the race of the pre-term neonate is white and the gender is male; providing ventilatory support to the pre-term neonate; determining that the fact that the pre-term neonate is a white male means the pre-term neonate is at a higher risk of mortality upon administration of inhaled nitric oxide compared to a black or other non-white pre-term neonate or a female pre-term neonate of the same gestational age; and, based on the determination of higher risk, either excluding the white, male, pre-term neonate from treatment with inhaled nitric oxide, or treating the white, male, pre-term neonate with inhaled nitric oxide, but using a treatment regimen shorter than typically used to treat the underlying condition. The ventilatory support may be continued after the pre-term neonate is excluded from treatment with inhaled nitric oxide, or may be substituted with ECMO at that point if the underlying condition involves hypoxia and if ECMO is appropriate for the patient. The ventilatory support may be high-frequency oscillatory ventilation. The white, male, pre-term neonate may also be treated with intravenous fluids, surfactant, and/or bicarbonate therapy. In some embodiments, the white, male, pre-term neonate is a candidate for inhaled nitric oxide at least in part because he is identified as being at risk of BPD. In other embodiments, the pre-term neonate is a candidate for inhaled nitric oxide because he has a condition that can lead to pulmonary hypertension, or is undergoing, or has just undergone, a procedure that can lead to pulmonary hypertension, such as cardiac surgery involving heart/lung bypass. Alternatively or in addition, the pre-term neonate may have pulmonary hypertension (e.g., associated with hypoxic respiratory failure) that can be relieved at least in part with inhaled nitric oxide.

In certain embodiments of any of the above methods, the neonates (in particular, the white, male, pre-term neonates) are not dependent on right-to-left shunting of blood, and/or do not have left ventricular dysfunction. The methods may include steps of carrying out standard tests (such as echocardiography) to determine whether the neonates have either or both of those conditions, prior to making a decision about administration of inhaled nitric oxide.

In certain embodiments of the above methods, the non-white neonates and female neonates, and also the white, male neonates who are more than 27 weeks postmenstrual age, who are not excluded from treatment with inhaled nitric oxide (because the above-described increased risk of mortality does not apply to them), are treated with inhaled nitric oxide as a means to prevent BPD. This treatment typically begins at least five days after birth of a pre-term (<30 weeks gestational age) neonate, e.g., five to fourteen days after birth, five to ten days after birth, five to seven days after birth, or five to 21 days after birth. A typical protocol for inhaled nitric oxide treatment of a pre-term neonate at risk of BPD involves administering decreasing concentrations of nitric oxide over the course of administration, beginning at 20 ppm. In certain embodiments, the initial dose of 20 ppm nitric oxide is decreased to 10, then 5, and then 2 ppm over the course of administration. In specific embodiments, the administration of nitric oxide is for at least 20 days, preferably at least 24 days, and can be up to 30 days. In a specific example of nitric oxide administration, the initial administration of 20 ppm nitric oxide lasts for a period of 48 to 96 hours, followed by a week at 10 ppm, a week at 5 ppm, and then a week at 2 ppm.

In certain embodiments, an alternative treatment for a pre-term neonate who is at risk of BPD, but who is not treated with inhaled nitric oxide because the pre- term neonate is a white male of less than 27 weeks gestational age, is one or more of any acceptable treatment, including but not limited to caffeine, vitamin A, supplemental oxygen, and one or more surfactants, antenatal steroid, etc. (Any of these alternative treatments (e.g., vitamin A or supplemental oxygen) may also be used in conjunction with inhaled nitric oxide for the prevention of BPD in appropriate patients.)

The invention also includes a system for use in administering inhaled nitric oxide gas. The system reduces the overall risk of mortality from inhalation of nitric oxide by providing instructions for use of nitric oxide that include a warning about a specific risk to a specific population of pre-term neonates. The system also includes a source of pharmaceutically acceptable nitric oxide gas for inhalation and a delivery device suitable for delivering nitric oxide gas from the source to a neonatal patient. The instructions may include, for example, a description of operating the system to deliver 20 ppm inhaled nitric oxide to treat a condition such as one or more of hypoxic respiratory failure or BPD or pulmonary arterial hypertension, a contraindication for neonates who are dependent on right to left shunting of blood, and/or a contraindication or warning regarding patients who have pre-existing left ventricular dysfunction. The instructions also include a warning that inhaled nitric oxide may produce an increased risk of death in white, male, pre-term infants of less than 27 weeks gestational age (and do not include a similar warning for pre-term infants of less than 27 weeks gestational age who are female or are not white). The latter warning may also specify that the white, male, pre-term infants of less than 27 weeks gestational age are ones who are at risk of developing BPD, may say the white, male, pre-term infants do not have pulmonary hypertension, and may be effective in reducing the overall risk of mortality from inhalation of nitric oxide compared to a similar system without the instructions, and may be effective in excluding a population of white, male, pre-term infants less than 27 weeks gestational age from treatment with inhaled nitric oxide, thereby reducing the overall risk of mortality resulting from inhaled nitric oxide treatment. In addition or instead, the instructions may communicate that, in a subset of white, male, pre-term infants less than 27 weeks gestational age, without pulmonary hypertension and at risk of developing BPD, there was seen in one study an imbalance in deaths in the INOmax treated group as compared to placebo, and may specify that the study used doses of 20 ppm for 3 days reduced to 10 ppm for 10 days and 5 ppm for remainder of 24 days.

In other embodiments, the white male neonate who is excluded is less than 30 weeks gestational age, or less than 29 weeks, 28 weeks, 26 weeks, 25 weeks, 24 weeks, or 23 weeks.

Also disclosed is a method of distributing a pharmaceutical product. This method includes supplying a source of nitric oxide or any material that can be converted to nitric oxide to a medical provider responsible for treating a plurality of neonatal patients who are candidates for treatment with inhaled nitric oxide (e.g., neonates with hypoxic respiratory failure and/or pulmonary hypertension, or those at risk of BPD), including pre-term neonates such as those of less than 27 weeks gestational age; informing the medical provider than a recommended dose of inhaled nitric oxide gas is 20 ppm nitric oxide; and providing a warning to the medical provider that the risk of mortality from inhalation of nitric oxide is higher in white, male, pre-term neonates less than 27 weeks gestational age (e.g., those who are at risk of BPD and so might otherwise be treated with inhaled nitric oxide to help prevent BPD) than in non-white pre-term neonates or female pre-term neonates of the same gestational age. In preferred embodiments, the warning is effective to cause a medical provider considering inhaled nitric oxide treatment in a white, male, pre-term neonate less than 27 weeks gestational age (e.g., for prevention of BPD) to forego use of inhaled nitric oxide in that pre-term neonate in order to avoid putting the pre-term neonate at increased risk of mortality, or otherwise to reduce the use of inhaled nitric oxide in such white, male, pre-term neonates, including those at risk of developing BPD. The medical provider may choose to substitute another appropriate treatment. The various instructions and warnings may be supplied to the medical provider in the form of a label attached to and/or shipped with the source of nitric oxide, or in materials otherwise supplied to the medical provider. The medical provider who is so informed may use the source of nitric oxide (or any material that can be converted to nitric oxide gas that is then delivered to and inhaled by a patient) to treat patients who are candidates for treatment with inhaled nitric oxide, including pre-term neonates. Prior to treating a given pre-term neonate, the medical provider may assess whether the pre-term neonate falls within one of the populations described in the instructions as being at increased risk of harm. This assessment includes determining whether the patient is a white, male, pre-term neonate of less than 27 weeks gestational age, and may include determining whether the patient is at risk of BPD. In accordance with the warning regarding that population, the medical provider will likely avoid treating such a patient with inhaled nitric oxide, and may utilize another suitable alternative treatment instead. If the patient is a non-white or a female, the warning does not apply, so the medical provider can treat such a patient with inhaled nitric oxide, even though the latter patient is of less than 27 weeks gestational age and can be characterized as at risk of BPD.

The method of distributing the pharmaceutical product may also include steps of manufacturing the source of nitric oxide gas before it is supplied to the medical provider. If the source of nitric oxide gas is a cylinder of compressed gas, it may be manufactured by a process comprising diluting nitric oxide gas with nitrogen gas to produce a compressed gaseous mixture of nitric oxide and nitrogen in a cylinder. The pharmaceutical product may instead include N₂O₄ (in liquid form) or nitrogen dioxide (in gaseous form); such a product is supplied in a form that is ultimately converted to nitric oxide at the patient's bedside. If the source of nitric oxide gas is a container containing compressed nitric oxide or nitrogen dioxide, the method may further comprise generating the nitric oxide or nitrogen dioxide and introducing it into the container in compressed form, prior to supplying it to the medical provider. By “supplying to the medical provider” is meant causing the source of nitric oxide gas to be delivered to the medical supplier, e.g., by transporting it from one site (such as the manufacturing location or a distributer or storage building) to another site (e.g., where the medical provider administers inhaled nitric oxide treatment to patients).

### DETAILED DESCRIPTION

Several exemplary embodiments of the claimed methods and systems are described below. It is to be understood that what is claimed is not limited to the details of set forth in the following description. The methods and systems are capable of other embodiments and of being practiced or being carried out in various ways.

INOmax® (nitric oxide) for inhalation is an approved drug product. The FDA-approved prescribing information for INOmax dated 2013 is attached as Appendix 1, and so forms part of the present disclosure, and also is incorporated by reference herein in its entirety. INOmax® is a selective pulmonary vasodilator, which, in conjunction with ventilatory support or other appropriate agents, is indicated for the treatment of term and near-term (>34 weeks gestation) neonates with hypoxic respiratory failure associated with clinical or echocardiographic evidence of pulmonary hypertension, where it improves oxygenation and reduces the need for extracorporeal membrane oxygenation. The recommended dose of INOmax for the approved indication is 20 ppm, maintained for up to 14 days or until the underlying oxygen desaturation has resolved. Weaning should occur gradually. Adverse reactions per the label include methemoglobinemia and nitrogen dioxide levels, both which can be dose dependent.

Inhaled nitric oxide may be administered via a delivery device such as INOmax DSIR®, INOmax® DS or INOvent®, each of which delivers operator-determined concentrations of nitric oxide in conjunction with a ventilator or breathing gas administration system after dilution with oxygen or an oxygen/air mixture. A nitric oxide delivery system includes a nitric oxide administration apparatus, a nitric oxide gas analyzer and a nitrogen dioxide gas analyzer.

The source of nitric oxide used in any of the presently disclosed methods can be a cylinder of compressed gas containing nitric oxide, typically as a mixture with an inert gas such as nitrogen or helium. Nitric oxide is generated by manufacturing the gases separately, mixing them in an appropriate ratio, and introducing them into an appropriate cylinder under pressure. The mixing may occur in two steps: first diluting bulk nitric oxide with nitrogen to a concentration of, e.g., 5,000 ppm or 28,600 ppm in interim cylinders, and then diluting that mixture further by introducing the mixture into the final cylinders and filling them with more nitrogen to produce a concentration of, e.g., 100 ppm or 800 ppm in the final cylinders. Care is taken not to introduce any water or oxygen into the cylinders. The cylinders are equipped with an appropriate valve, shipped to the point of use, and attached to a delivery device (as described above) to facilitate inhalation of the gas by the patient.

The source of nitric oxide can instead be a nitric oxide-generating device that generates nitric oxide from a suitable nitrogen source, such as air (see for reference U.S. Pat. No. 5,396,882, incorporated herein by reference) or nitrogen dioxide. The source of nitrogen dioxide can be, for example, a canister of compressed nitrogen dioxide gas or a container of N₂O₄ (which, when treated under appropriate conditions, will give off nitrogen dioxide). Manufacturing a source of nitrogen dioxide includes steps of compressing nitrogen dioxide gas into a suitable container or introducing N₂O₄ in liquid form into a suitable container. The container is supplied in a device that includes a filter containing a reducing agent or antioxidant, such as ascorbic acid, which reduces the nitrogen dioxide to form nitric oxide at the patient's bedside. At the point of administration, such a nitric oxide-generating device is typically attached to a gas-delivery device (such as a ventilator) to facilitate inhalation of the newly formed nitric oxide gas by the patient.

Inhaled nitric oxide is contraindicated in the treatment of neonates known to be dependent on right-to-left shunting of blood.

Patients with left ventricular dysfunction who are treated with nitric oxide may experience pulmonary edema, increased pulmonary capillary wedge pressure, worsening of left ventricular dysfunction, systemic hypotension, bradycardia and cardiac arrest. If this occurs, the medical provider is advised to discontinue inhaled nitric oxide while providing symptomatic care. The medical provider may choose to forego use of inhaled nitric oxide altogether in patients who are determined to have pre-existing left ventricular dysfunction, or may administer inhaled nitric oxide while monitoring carefully for adverse events related to the left ventricular dysfunction (such as pulmonary edema), and discontinuing use of the gas only if necessary.

“Neonate” or “neonatal” refers to an infant within 4 weeks of birth.

“Pre-term neonate” or “preterm neonate” refers to a neonate who is less than 34 weeks gestational age.

Despite multiple trials indicating the safety of inhaled nitric oxide in full-term, near-term, and pre-term neonates (several clinical trials in the latter group are summarized in the “Data Review and Bibliography—Use in Premature Neonates” document attached as Appendix 2, which forms part of the present disclosure and also is incorporated by reference in its entirety), it has been unexpectedly discovered that there is a specific neonatal patient population that is at increased risk of mortality (i.e., death) if treated with inhaled nitric oxide, compared to the same population that does not receive inhaled nitric oxide. The increased mortality rate was unexpectedly observed in white, male, pre-term neonates of gestational age less than 27 weeks who were being treated with inhaled nitric oxide because they were at risk of BPD, on the theory that inhaled nitric oxide may be useful in preventing (reducing the incidence of) BPD. The increased mortality rate was not observed in non-white, male pre-term neonates of gestational age less than 27 weeks, or in female pre-term neonates of any race who were of the same gestational age. Once an individual white, male pre-term neonate born at less than 27 weeks gestation age reaches a point corresponding to 27 weeks postmenstrual age and at least 5 days after birth, he can safely receive inhaled nitric oxide.

Alternative preventative treatments for white, male, pre-term neonates initially identified as candidates for inhaled nitric oxide treatment intended for the prevention of BPD, but who are now excluded from that preventative treatment because of the newly discovered risk of mortality, include one or more of caffeine, vitamin A, supplemental oxygen, and/or surfactant. Oxygen is generally administered at 21-60% of the inhaled gas, but can be administered at a higher level if necessary. It is considered to be supplemental oxygen if the patient's inhalation gas contains a level of oxygen higher than in air, i.e., at least 22% oxygen (e.g., 22-60%), and up to 90%, 99% or even 100% oxygen. Caffeine therapy can be, e.g., 20-25 mg/kg every 24 hours as citrate orally or IV (intravenous) over 10 minutes, beginning 24 hours after loading dose. Alternatively, 18-30 mg/kg every 12 or more hours may be administered. Vitamin A therapy includes, for example, 7,500-15,000 IU intramuscularly daily for up to 10 days. Surfactant treatment generally depends on the type of surfactant being administered, but is typically administered in three doses: first dose is 3 cc/kg, then if FiO₂ (fraction of inspired oxygen) six hours after the first dose is greater than 60%, the second dose of 3 cc/kg is administered. If FiO₂ six hours after the second dose is greater than 40%, then the third dose of 3 cc/kg is administered.

Certain risk calculators may be used to assist in identifying if a patient is at risk of BPD. Risk calculator software known in the art may take into account a pre-term neonate's gestational age, weight and ventilator support needs, among other factors. Such a risk calculator may help a clinician or therapist to assist in determining if a given pre-term neonate is at risk of developing BPD. See, e.g., Laughon et al., (2011) Am J Respir Crit Care Med 183:1715-1722.

As in other situations where the race of the patient has an effect on treatment options, determining whether the patient is white or non-white is generally accomplished by visual assessment coupled with querying the patient's parent or other relative, and/or reviewing the patient's records. Self-identification of the patient's race by the patient's biological mother or father is typically the means by which race is determined for this purpose. Biological markers (such as genetic markers) indicative of race may also be useful, where such markers have been validated as accurate predictors of whether a patient is white vs. non-white. As the heightened risk in white males, compared to non-white males, is theorized to be due to a recessive trait present in white males and not in non-white males, a male pre-term neonate of mixed white and non-white ancestry is (according to this theory) likely to carry the dominant trait that is protective in non-whites. Thus, if there is a question as to how to categorize a male pre-term neonate of mixed white and non-white ancestry for purposes of the presently claimed methods, a physician may choose to categorize the pre-term neonate as non-white and thus eligible for treatment with inhaled nitric oxide.

In general, administration of inhaled nitric oxide to pre-term neonates that have been identified as at risk for developing BPD and/or as likely to have a therapeutic response to the inhaled nitric oxide should begin no less than five days after birth. Treatment of these pre-term neonates may begin at some point within the period of 5 to 14 days after birth, 7 to 21 days after birth, 5 to 21 days after birth, or 5 to 7 days after birth. Administration of inhaled nitric oxide for this purpose generally continues for 20-30 days after it is initiated. In specific treatment protocols, administration of inhaled nitric oxide may be continued for at least 20 days, 21 days, 22 days, 23 days, 24 days, 25 days, 26 days, 27 days, 28 days, 29 days or 30 days.

### EXAMPLES

A multi-center, double blind, placebo-controlled randomized clinical trial designated the “BPD-301” study was conducted to examine the efficacy of inhaled nitric oxide in preventing BPD in preterm neonates less than 30 weeks gestational age and less than 1250 grams who required mechanical ventilation or positive pressure support. Patients were identified as (1) American Indian or Alaska Native, (2) Asian, (3) Black, (4) Hispanic, (5) White, or (6) Other. Continuous inhaled nitric oxide or placebo gas administration into the inspiratory limb of the ventilator circuit in the mechanically ventilated subject was accomplished using an INOvent® delivery device. The placebo group received the same dose of nitrogen that the active or treatment group received of inhaled nitric oxide. Administration of inhaled nitric oxide or placebo was begun at a point during the window of time 5 to 14 days after the subject's birth and continued for 24 days thereafter. The starting dose was 20 ppm. A dose reduction schedule was followed: after 72 hours of treatment at 20 ppm, reducing to 10 ppm, and then reducing to 5 ppm on day 10. If an infant was extubated before 24 days, therapy was continued via nasal continuous positive airway pressure or nasal cannula to complete the protocol. 451 infants less than 30 weeks of gestation age were included in this trial, of which 449 were randomized and 2 were not randomized. There was a 1:1 randomization with 220 infants treated with placebo and 229 treated with inhaled nitric oxide.

It is believed that pre-term neonates that enrolled in and completed the BPD 301 study did not have pulmonary hypertension, since one of the exclusionary criteria was prior treatment with inhaled nitric oxide for any reason, and pulmonary hypertension in infants is routinely treated with inhaled nitric oxide. In order to enroll in the BPD 301 study and receive inhaled nitric oxide for the prevention of BPD, a pre-term neonate could not have already previously been administered inhaled nitric oxide. If a pre-term neonate exhibited at birth characteristics associated with pulmonary hypertension, he or she would have likely been treated with commercial inhaled nitric oxide, and if treated with commercial inhaled nitric oxide, he or she would not have been eligible for the study.

Additionally, if the pre-term neonate had undiagnosed pulmonary hypertension and happened to be placed on placebo (nitrogen) for purposes of the BPD-301 study, the pre-term neonate's hypoxic respiratory failure would have worsened and would have likely required immediate treatment with commercial inhaled nitric oxide. This administration of commercial inhaled nitric oxide would have failed the subject from the trial, both reducing the power of the trial and eliminating the subject from the study analysis.

A synopsis of the BPD-301 protocol follows.

Name of Sponsor/Company: INO Therapeutics LLC
Name of Investigational Product: INOmax ® (nitric oxide) for inhalation
Name of Active Ingredient: Nitric Oxide for Inhalation
Title of Study: Inhaled nitric oxide for the prevention of bronchopulmonary dysplasia (BPD) in
preterm infants requiring mechanical ventilation or positive pressure support on days 5 to 14
after birth
Study center(s): Approximately 25

| | |
|---|---|
| Studied period (years): | Phase of development: 3 |
| Estimated date first subject enrolled: Dec. 30, 2009 | |
| Estimated date last subject completed: Aug. 2, 2012 | |
| Estimated end of follow up: Aug. 2, 2014 | |

Objectives:
Primary:
The primary objective of this trial is to examine the efficacy of inhaled nitric oxide (iNO) in
preterm infants <30 weeks gestational age (GA) and <1250 grams who require mechanical
ventilation or positive pressure support on days 5 to 14 after birth.
Secondary:
The secondary objectives are to examine the safety of iNO on premature infants as measured by
survival, adverse events, and outcomes at 18 months to 24 months of age, to evaluate the
pharmacokinetics (PK) of iNO in preterm infants, and to measure the pharmacoeconomic (PE)
impacts of iNO use on the management of preterm infants. Assessment will be:
To examine the safety of inhaled nitric oxide (iNO) on premature infants as measured by
survival and adverse events.
To evaluate the pharmacokinetics (PK) of nitric oxide (NO) in preterm infants using plasma
nitrite and nitrate (NOx) as surrogates
To evaluate the relationship between NOx and methemoglobin (MetHb)
To explore the relationship between NO exposure and clinical outcomes
To evaluate the economic implications of using nitric oxide for inhalation (iNO) to prevent
BPD
Methodology:
Multi-center, double blind, placebo-controlled, randomized clinical trial. Infants who meet all
enrollment criteria at any point during days 5 to 14 after birth will be randomized to inhaled NO
starting at 20 ppm, or matching placebo, by means of a blinded INOvent ® delivery device. All
infants will receive 24 days of therapy, following a dose reduction schedule. Infants who are
extubated before 24 days will continue therapy via nasal continuous positive airway pressure
(CPAP) or nasal cannula to complete 24 days' of therapy. The primary outcome measure will be
survival without BPD at 36 weeks postmenstrual age using a physiologic assessment of BPD. Sparse
pharmacokinetic (PK) samples will be collected from participating infants (3 samples each) on a
randomized schedule. This study will include a 12 month and 18 to 24 month follow-up visits to
assess safety and efficacy. All infants will be followed up to 18 to 24 months of corrected age
for adverse events, somatic growth and neurologic development.
Number of subjects (planned): 450
Diagnosis and main criteria for inclusion:
Inclusion:
Preterm infants who are:
1. <1250 grams at birth
2. <30 weeks gestational age
3. 5 to 14 days of age (inclusive) at the time of entry
4. Requiring mechanical ventilation or for those infants ≤800 grams, positive pressure
support (including CPAP) for primary respiratory insufficiency on days 5 to 14 days of age (inclusive)
Exclusion:
1. Preterm infants with life-threatening anomalies (cranial, cardiac, thoracic,
chromosomal) or congenital diaphragmatic hernia with lung hypoplasia, or any subject who will
not receive complete intensive care
2. Preterm infants with bilateral Grade 4 intraventricular hemorrhage (IVH)
3. Subjects who are dependent on right to left shunting to maintain the systemic circulation
4. Preterm infants who received prior iNO therapy
5. Use of another investigational agent
Investigational product, dosage and mode of administration: Nitric oxide for inhalation (or
matching placebo) starting at 20 ppm (and following a dose reduction schedule) will be
administered continuously into the inspiratory limb of the ventilator circuit in mechanically
ventilated subject using a blinded version of the INOvent ® delivery system, or by nasal CPAP or
nasal cannula, as needed to complete 24 days of therapy.
Duration of treatment: Study drug will be weaned from 20 ppm to 10 ppm after 72 hours of
treatment. The next wean will be to 5 ppm on Day 10. If the infant deteriorates after any dose
reduction, he/she may be put back on the previous treatment and weaning re-attempted as
tolerated. Duration of treatment is 24 days.
Reference therapy, dosage and mode of administration: Placebo consisting of 100% Grade 5
Nitrogen (N₂) gas will be administered in a blinded manner identical to that of the active
treatment.
Criteria for evaluation:
Efficacy:
Survival without BPD at 36 weeks postmenstrual age using a physiologic assessment
Severity of BPD (as defined by FiO₂ requirement at week 36) among survivors as
determined by the level of support needed to remain SaO₂ ≥ 90%
Status at 40 and 44 weeks postmenstrual age
Hospitalization
Need for oxygen
Need for airway pressure support
Need for mechanical ventilation
Days of air pressure support for lung disease (birth hospitalization)
Days of hospitalization (birth hospitalization)
Use of postnatal corticosteroids for respiratory insufficiency (including dose and
duration of therapy) (birth hospitalization)
Use of respiratory medications, oxygen, and days of all hospitalizations at 12 months
and 18 to 24 months of corrected age.
Safety: Examine the safety of iNO in preterm infants at risk of BPD.
Incidence of methemoglobinemia and elevated nitrogen dioxide concentrations
Incidence and types of adverse events including:
Adverse events of interest will include intracranial hemorrhage of any type,
patent ductus arteriosus requiring intervention, necrotizing enterocolitis, intestinal perforations,
air leaks of any type, pulmonary hemorrhage, retinopathy of prematurity and sepsis of any type.
Incidence of death after 36 weeks postmenstrual to 12 month and 18 to 24 month of
corrected age, stratified by gestational age at birth.
Long-term neurodevelopmental outcome assessed by age appropriate developmental
assessments at 18 to 24 months of corrected age. Neurologic assessments at 18 to 24 months
will include the cognitive and language components of the Bayleys-3, a structured neurologic
examination, and a parental questionnaire.
Pharmacoeconomics (PE): Assessments of resource utilization will be made to determine if
INOmax therapy is cost effective or cost saving.
Pharmacokinetics (PK): Concentration data of NOx from sparse samples will be pooled to
evaluate the following PK parameters. Individual parameters may be estimated based on
simulated results. Demographic covariates may be evaluated using a population PK analysis
approach if sufficient data is available.
Cₘₐₓ: observed maximum plasma concentration
Tₘₐₓ: time at Cₘₐₓ
AUC: area under plasma concentration-time curve from 0 to last quantifiable time
point (AUC₀₋ₜ) and from 0 to infinity (AUC_{0-inf})
T_{1/2}: terminal half-life
Vₛₛ: volume of distribution at steady state
CL: total clearance
PK Analyses:
Linear correlation between NOx and MetHb will be evaluated.
Exposure-response relationship (PK vs. clinical outcomes) may be evaluated when
sufficient data available.
Statistical methods:
All efficacy analyses will be performed on an intent-to-treat (ITT) population that consists of all
randomized subjects. Safety analyses will be performed on a safety population that includes all
randomized subjects who receive study drug. The primary efficacy endpoint is survival without
BPD at 36 weeks GA. The primary method of analysis will use the generalized estimating
equation (GEE) for logistic regression. In addition, the Cochran-Mantel-Haenszel (CMH) test
will be performed to further evaluate the primary endpoint. For the secondary efficacy
endpoints, GEE for logistic regression will be used to analyze the binary response variables
and mixed-effects model with repeated-measures will be used to analyze continuous variables.
Additional statistical analyses will be performed using CMH test and van Elteren test as
appropriate. All statistical tests will be two-sided with a significance level of 0.05.

A safety analysis of the randomized population in the BPD-301 study revealed an unexpected anomalous finding in a subpopulation of white, male, preterm infants of less than 27 weeks. Data from this study indicate that white, male, preterm infants of less than 27 weeks gestational age are at significant risk of death when treated with inhaled nitric oxide for the prevention of BPD, as compared to placebo. The placebo and treatment groups were matched for the following characteristics: gestational age, age at the start of treatment, sex, length at birth, weight, head circumference, respiratory severity scores, apgar scores, and physical exam results. There were no anomalous findings to indicate that there was any particular reason, other than the inhaled nitric oxide itself, that caused this result.

The overall mortality rate in the trial was low. The mortality rate was not significantly different between inhaled nitric oxide and placebo groups (11.35% vs. 8.64%, p=0.436), evaluated with Fisher's exact test. See Table 1. Analysis of the mortality rates in different race groups demonstrated that, among the white population, the inhaled nitric oxide-treated pre-term infants had an unexpected higher mortality rate than those from the placebo group [Table 1].

TABLE 1
SUMMARY OF MORTALITY BY RACE

| | PLACEBO | INO | |
|---|---|---|---|
| | N = 220 | N = 229 | |
| | n (%) | n (%) | P-VALUE ⁽¹⁾ |
| ALL | 19 (8.64%) | 26 (11.35%) | 0.436 |
| American Indian | 1 (0.45%) | 0 (0.00%) | |
| or Alaska Native | | | |
| Asian | 2 (0.91%) | 0 (0.00%) | |
| Black | 9 (4.09%) | 6 (2.62%) | |
| Hispanic | 4 (1.82%) | 3 (1.31%) | |
| Native Hawaiian or | 0 (0.00%) | 0 (0.00%) | |
| other Pacific Islander | | | |
| Other | 1 (0.45%) | 2 (0.87%) | |
| White | 2 (0.91%) | 15 (6.55%) | |

⁽¹⁾ Fisher's exact test.

In the subgroup of white preterm infants of less than 27 weeks, deaths were a statistically significant 15 (12.5%) for the treatment group versus only 2 (2.4%) in the placebo group [Tables 1 and 2]. There was no similar difference in mortality between treatment and placebo groups for any other racial category [Table 1].

TABLE 2
MORTALITY FOR WHITE PRETERM INFANTS
LESS THAN 27 WEEKS GESTATIONAL AGE

| | PLACEBO | INO | |
|---|---|---|---|
| | N = 84 | N = 120 | |
| ALIVE | n (%) | n (%) | P-VALUE ⁽¹⁾ |
| Yes | 82 (97.6%) | 105 (87.5%) | 0.010 |
| No | 2 (2.4%) | 15 (12.5%) | |

⁽¹⁾ Fisher's exact test.

Additional analyses were performed to assess role of gender on mortality in the subset population of white infants less than 27 weeks gestational age. The mortality rate of the inhaled nitric oxide-treated white infants was significantly higher in the male group than in the female group (21.4% (3 of 15) vs. 4.7% (12 of 15), p=0.011) [Table 3]. All the white male infants that died were less than 27 weeks gestational age. One white male in the placebo group died and one white female in the placebo group died. Out of the 84 total placebo population, 37 were female and 47 were male. At the end of the study evaluation period, there was a highly statistically significant finding of death/mortality in the active/treatment arm as compared to the placebo. In sum, the BPD 301 trial unexpectedly identified an increased risk of mortality for white males less than 27 weeks gestational age, treated with inhaled nitric oxide for the prevention of BPD, than was shown in treated white females of the same gestational age. This gender discrepancy and unexpected mortality risk were not witnessed in other race groups.

TABLE 3
MORTALITY BY GENDER FOR INHALED NITRIC
OXIDE-TREATED WHITE INFANTS LESS
THAN 27 WEEKS GESTATIONAL AGE

| | MALE | FEMALE | |
|---|---|---|---|
| | N = 56 | N = 64 | |
| ALIVE | n (%) | n (%) | P-VALUE ⁽¹⁾ |
| Yes | 44 (78.6%) | 61 (95.3%) | 0.011 |
| No | 12 (21.4%) | 3 (4.7%) | |

⁽¹⁾ Fisher's exact test.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the inventions herein have been described with reference to particular embodiments, it is to be understood that these embodiments are merely illustrative of the principles and applications of the present inventions. It will be apparent to those skilled in the art that various modifications and variations can be made to the methods and system of the present invention without departing from the spirit and scope of the inventions. Thus, it is intended that the present inventions include modifications and variations that are within the scope of the appended claims and their equivalents.

1-42. (canceled)

43. A method of treatment comprising
(a) identifying a plurality of neonates as being candidates for treatment with inhaled nitric oxide;
(b) determining that the race of a first neonate of the plurality is non-white;
(c) administering inhaled nitric oxide to the first neonate;
(d) determining that the race of a second neonate of the plurality is white;
(e) determining that the gender of the second neonate is male;
(f) determining that the second neonate is of less than 27 weeks post-menstrual age;
(g) determining that the fact that the second neonate is a white male of less than 27 weeks post-menstrual age means the second neonate is at a higher risk of mortality upon administration of inhaled nitric oxide compared to a female or non-white male neonate of the same post-menstrual age; and
(h) administering to the second neonate a treatment that comprises mechanical ventilation or positive airway pressure support while excluding the second neonate from treatment with inhaled nitric oxide, the exclusion from treatment with inhaled nitric oxide being based on the determination of (g).

44. The method of claim 43, wherein the second neonate does not have pulmonary hypertension.

45. The method of claim 43, wherein the second neonate is identified as being at risk of developing bronchopulmonary dysplasia (BPD).

46. The method of claim 43, wherein determining that the race of the second neonate is white comprises (i) visually assessing the race of the second neonate, or (ii) querying a relative of the second neonate about the race of the second neonate, or (iii) both (i) and (ii).

47. The method of claim 43, wherein the second neonate does not have left ventricular dysfunction and is not identified as being dependent on right-to-left shunting of blood.

48. The method of claim 43, wherein the treatment administered to the second neonate further comprises administration of a surfactant.

49. The method of claim 43, wherein the treatment administered to the second neonate further comprises extra-corporeal membrane oxygenation (ECMO).

50. The method of claim 63, wherein the second neonate is treated with ECMO after being excluded from treatment with inhaled nitric oxide.
